SEQUENCE LISTING (50 sequences)

SEQ ID NO: 1 - protein, 10 aa, Artificial Sequence
  source: /note=“Description of Artificial Sequence Synthetic peptide”
GYIFSNYWIQ

SEQ ID NO: 2 - protein, 17 aa, Artificial Sequence
  source: /note=“Description of Artificial Sequence Synthetic peptide”
EILPGSGSTEYTENFKD

SEQ ID NO: 3 - protein, 13 aa, Artificial Sequence
  source: /note=“Description of Artificial Sequence Synthetic peptide”
YFFGSSPNWYFDV

SEQ ID NO: 4 - protein, 11 aa, Artificial Sequence
  source: /note=“Description of Artificial Sequence Synthetic peptide”
GASENIYGALN

SEQ ID NO: 5 - protein, 7 aa, Artificial Sequence
  source: /note=“Description of Artificial Sequence Synthetic peptide”
GATNLAD

SEQ ID NO: 6 - protein, 9 aa, Artificial Sequence
  source: /note=“Description of Artificial Sequence Synthetic peptide”
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  source: /note=“Description of Artificial Sequence Synthetic peptide”
GASENIYHALN

SEQ ID NO: 18 - protein, 17 aa, Artificial Sequence
  source: /note=“Description of Artificial Sequence Synthetic peptide”
EILPGSGHTEYTENFKD

SEQ ID NO: 19 - protein, 10 aa, Artificial Sequence
  source: /note=“Description of Artificial Sequence Synthetic peptide”
GHIFSNYWIQ

SEQ ID NO: 20 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, Artificial Sequence
  source: /note=“Description of Artificial Sequence Synthetic peptide”
SYAIS

SEQ ID NO: 22 - protein, 17 aa, Artificial Sequence
  source: /note=“Description of Artificial Sequence Synthetic peptide”
GIGPFFGTANYAQKFQG

SEQ ID NO: 23 - protein, 7 aa, Artificial Sequence
  source: /note=“Description of Artificial Sequence Synthetic peptide”
DTPYFDY

SEQ ID NO: 24 - protein, 11 aa, Artificial Sequence
  source: /note=“Description of Artificial Sequence Synthetic peptide”
SGDSIPNYYVY

SEQ ID NO: 25 - protein, 7 aa, Artificial Sequence
  source: /note=“Description of Artificial Sequence Synthetic peptide”
DDSNRPS

SEQ ID NO: 26 - protein, 11 aa, Artificial Sequence
  source: /note=“Description of Artificial Sequence Synthetic peptide”
QSFDSSLNAEV

SEQ ID NO: 27 - protein, 116 aa, Artificial Sequence
  source: /note=“Description of Artificial Sequence Synthetic polypeptide”
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, Artificial Sequence
  source: /note=“Description of Artificial Sequence Synthetic polypeptide”
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, Artificial Sequence
  source: /note=“Description of Artificial Sequence Synthetic peptide”
NYIS

SEQ ID NO: 30 - protein, 17 aa, Artificial Sequence
  source: /note=“Description of Artificial Sequence Synthetic peptide”
IIDPDDSYTEYSPSFQG

SEQ ID NO: 31 - protein, 8 aa, Artificial Sequence
  source: /note=“Description of Artificial Sequence Synthetic peptide”
YEYGGFDI

SEQ ID NO: 32 - protein, 11 aa, Artificial Sequence
  source: /note=“Description of Artificial Sequence Synthetic peptide”
SGDNIGNSYVH

SEQ ID NO: 33 - protein, 7 aa, Artificial Sequence
  source: /note=“Description of Artificial Sequence Synthetic peptide”
KDNDRPS

SEQ ID NO: 34 - protein, 9 aa, Artificial Sequence
  source: /note=“Description of Artificial Sequence Synthetic peptide”
GTYDIESYV

SEQ ID NO: 35 - protein, 116 aa, Artificial Sequence
  source: /note=“Description of Artificial Sequence Synthetic polypeptide”
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, Artificial Sequence
  source: /note=“Description of Artificial Sequence Synthetic polypeptide”
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 6 aa, Artificial Sequence
  source: /note=“Description of Artificial Sequence Synthetic peptide”
SSYYVA

SEQ ID NO: 38 - protein, 17 aa, Artificial Sequence
  source: /note=“Description of Artificial Sequence Synthetic peptide”
AIYTGSGATYKASWAKG

SEQ ID NO: 39 - protein, 13 aa, Artificial Sequence
  source: /note=“Description of Artificial Sequence Synthetic peptide”
DGGYDYPTHAMHY

SEQ ID NO: 40 - protein, 11 aa, Artificial Sequence
  source: /note=“Description of Artificial Sequence Synthetic peptide”
QASQNIGSSLA

SEQ ID NO: 41 - protein, 7 aa, Artificial Sequence
  source: /note=“Description of Artificial Sequence Synthetic peptide”
GASKTHS

SEQ ID NO: 42 - protein, 12 aa, Artificial Sequence
  source: /note=“Description of Artificial Sequence Synthetic peptide”
QSTKVGSSYGNH

SEQ ID NO: 43 - protein, 123 aa, Artificial Sequence
  source: /note=“Description of Artificial Sequence Synthetic polypeptide”
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 44 - protein, 110 aa, Artificial Sequence
  source: /note=“Description of Artificial Sequence Synthetic polypeptide”
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 45 - protein, 451 aa, Artificial Sequence
  source: /note=“Description of Artificial Sequence Synthetic polypeptide”
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 46 - protein, 217 aa, Artificial Sequence
  source: /note=“Description of Artificial Sequence Synthetic polypeptide”
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 120 aa, Artificial Sequence
  source: /note=“Description of Artificial Sequence Synthetic polypeptide”
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 48 - protein, 107 aa, Artificial Sequence
  source: /note=“Description of Artificial Sequence Synthetic polypeptide”
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 49 - protein, 447 aa, Artificial Sequence
  source: /note=“Description of Artificial Sequence Synthetic polypeptide”
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Artificial Sequence
  source: /note=“Description of Artificial Sequence Synthetic polypeptide”
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0227851 A1
(43) Pub. Date: Jul. 21, 2022

(54) METHODS OF TREATING VITILIGO USING AN ANTI-C5 ANTIBODY

(71) Applicant: Alexion Pharmaceuticals, Inc., Boston, MA (US)

(72) Inventor: Richard Alexander Wells, Toronto (CA)

(73) Assignee: Alexion Pharmaceuticals, Inc., Boston, MA (US)

(21) Appl. No.: 17/612,368

(22) PCT Filed: May 20, 2020
(86) PCT No.: PCT/US2020/033732

§ 371 Date: Nov. 18, 2021

Related U.S. Application Data
(60) Provisional application No. 62/852,340, filed on May 24, 2019.

(51) Int. Cl. C07K 16/18; A61P 17/00; A61P 7/00

(52) U.S. Cl. CPC C07K 16/18; A61K 2039/545; A61P 7/00; A61P 17/00

(57) ABSTRACT

Provided are methods for clinical treatment of vitiligo using an anti-CS antibody or antigen binding fragment thereof.

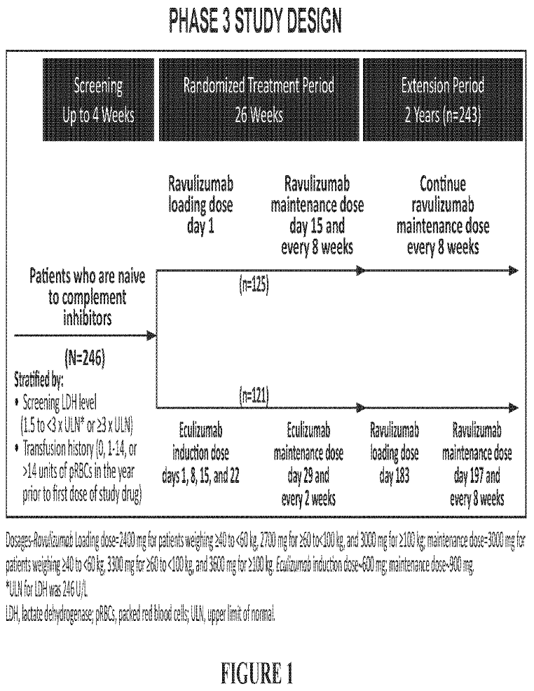

### RELATED APPLICATIONS

This application claims priority to U.S. Provisional Application No. 62/852,340, filed on May 24, 2019, the entire contents of which is incorporated herein by reference.

### SEQUENCE LISTING

The instant application contains a Sequence Listing which has been submitted electronically in ASCII format and is hereby incorporated by reference in its entirety. Said ASCII copy, created on May 14, 2020, is named AXJ-265PC_SL.txt and is 58,762 bytes in size.

### BACKGROUND

Vitiligo is an acquired skin disorder affecting 0.5-2% of the population worldwide, characterized by solitary or multiple well-defined depigmented macules. Vitiligo causes the loss of skin color in blotches. The extent and rate of color loss from vitiligo is unpredictable and can affect the skin on any part of the body, hair and/or the inside of the mouth. Normally, the color of hair and skin is determined by melanin. Vitiligo occurs when the cells that produce melanin die or stop functioning.

Despite numerous studies across decades and all over the world, the pathogenesis of vitiligo remains elusive. Many theories have been considered over time, including genetic predisposition, a neural-based theory, an autoimmune hypothesis, a reactive oxygen species model, zinc-α2-glycoprotein deficiency hypothesis, a viral theory, an intrinsic theory, as well as biochemical, molecular and cellular alteration theories to account for the loss of functioning melanocytes in vitiligo (Mohammed, G. et al., World J. Clin. Cases, 3:221-30. 2015). Cellular and humoral autoimmune responses have also been implicated in the destruction of melanocytes. Anti-melanocyte antibodies are present in the serum of vitiligo patients and have been shown to damage melanocytes in vitro via activation of the classic complement pathway and in vivo by passive immunization of nude mice (Norris D. et al., J. Invest. Dermatol., 90:783-9, 1988; Kemp, E. et al., Autoimmun. Rev., 6:138-42, 2007). An in vitro study reported that lysis of melanocytes due to antibody-mediated complement attack is increased when complement regulatory molecules are blocked (van den Wijngaard, R. et al., Br. J. Dermatol., 146:80-7, 2002), and another demonstrated the reduction of the CRM in vitiligo skin (Venneker, G. et al., Immunobiology. 198:476-84, 1998). Furthermore, in vitro and in vivo studies showed a destruction of melanoma by IgG from vitiligo patients. However, vitiligo is a multifactorial disease involving the interplay of several factors and further research is needed to clarify the interaction of numerous factors to better understand its pathogenesis.

Treating vitiligo can restore color to the affected skin, but it does not prevent continued loss of skin color or a recurrence. Moreover, treating vitiligo is a challenge for dermatologists since there is no definitive curative treatment. Existing treatment options for vitiligo, include topical or systemic corticosteroids, calcineurin inhibitors, vitamin D analogues, systemic therapies, surgery and phototherapy.

Despite existing treatment options, however, vitiligo is a psychologically debilitating and disfiguring disease, and many patients are refractory (e.g., resistant) to treatment. Accordingly, it is an object herein to provide improved methods for treating patients with vitiligo, including refractory vitiligo.

### SUMMARY

Provided herein are compositions and methods for treating vitiligo (e.g., refractory vitiligo) in a human patient, comprising administering to the patient an anti-C5 antibody, or antigen binding fragment thereof. In some embodiments, the anti-C5 antibody, or antigen binding fragment thereof, is administered (or is for administration) according to a particular clinical dosage regimen (i.e., at a particular dose amount and according to a specific dosing schedule). In some embodiments, the patient has vitiligo and Paroxysmal Nocturnal Hemoglobinuria (PNH).

Any suitable anti-C5 antibody, or antigen binding fragment thereof, can be used in the methods described herein. An exemplary anti-C5 antibody is eculizumab. Eculizumab (also known as SOLIRIS®) is an anti-C5 antibody comprising heavy chain CDR1, CDR2 and CDR3 domains having the sequences set forth in SEQ ID NOs: 1, 2, and 3, respectively, and light chain CDR1, CDR2 and CDR3 domains having the sequences set forth in SEQ ID NOs: 4, 5, and 6, respectively. Eculizumab comprises a heavy chain variable region having the amino acid sequence set forth in SEQ ID NO: 7 and a light chain variable region having the amino acid sequence set forth in SEQ ID NO: 8. Eculizumab comprises a heavy chain comprising the amino acid sequence set forth in SEQ ID NO:10 and a light chain having the amino acid sequence set forth in SEQ ID NO:11.

Another exemplary anti-C5 antibody is ravulizumab (also known as ULTOMIRIS®, ALXN1210 and antibody BNJ441) comprising the heavy and light chains having the sequences shown in SEQ ID NOs:14 and 11, respectively, or antigen binding fragments and variants thereof. In other embodiments, the antibody comprises the heavy and light chain complementarity determining regions (CDRs) or variable regions (VRs) of ravulizumab. Accordingly, in one embodiment, the antibody comprises the CDR1, CDR2, and CDR3 domains of the heavy chain variable (VH) region of ravulizumab having the sequence shown in SEQ ID NO:12, and the CDR1, CDR2 and CDR3 domains of the light chain variable (VL) region of ravulizumab having the sequence shown in SEQ ID NO:8. In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively. In another embodiment, the antibody comprises VH and VL regions having the amino acid sequences set forth in SEQ ID NO:12 and SEQ ID NO: 8, respectively. In another embodiment, the antibody comprises a heavy chain constant region as set forth in SEQ ID NO:13.

In another embodiment, the antibody comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

In another embodiment, the antibody binds to human C5 at pH 7.4 and 25° C. with an affinity dissociation constant (K_{D}) that is in the range 0.1 nM≤K_{D}≤1 nM. In another embodiment, the antibody binds to human C5 at pH 6.0 and 25° C. with a K_{D}≥10 nM. In yet another embodiment, the [(K_{D} of the antibody or antigen-binding fragment thereof for human C5 at pH 6.0 and at 25° C.)/(K_{D} of the antibody or antigen-binding fragment thereof for human C5 at pH 7.4 and at 25° C.)] of the antibody is greater than 25.

Another exemplary anti-C5 antibody is described in U.S. Pat. Nos. 8,241,628 and 8,883,158. In one embodiment, the antibody, or antigen binding fragment thereof, comprises heavy chain CDR1, CDR2 and CDR3 domains having the sequences set forth in SEQ ID NOs: 21, 22, and 23, respectively, and light chain CDR1, CDR2 and CDR3 domains having the sequences set forth in SEQ ID NOs: 24, 25, and 26, respectively. In another embodiment, the antibody, or antigen binding fragment thereof, comprises the VH region having the sequence set forth in SEQ ID NO:27, and the VL region having the sequence set forth in SEQ ID NO:28.

Another exemplary anti-C5 antibody is also described in U.S. Pat. Nos. 8,241,628 and 8,883,158. In one embodiment, the antibody, or antigen binding fragment thereof, comprises heavy chain CDR1, CDR2 and CDR3 domains having the sequences set forth in SEQ ID NOs: 29, 30, and 31, respectively, and light chain CDR1, CDR2 and CDR3 domains having the sequences set forth in SEQ ID NOs: 32, 33, and 34, respectively. In another embodiment, the antibody comprises the VH region having the sequence set forth in SEQ ID NO: 35, and the VL region having the sequence set forth in SEQ ID NO: 36.

Another exemplary anti-C5 antibody is described in US2016/0176954A1. In one embodiment, the antibody, or antigen binding fragment thereof, comprises heavy chain CDR1, CDR2 and CDR3 domains having the sequences set forth in SEQ ID NOs: 37, 38, and 39, respectively, and light chain CDR1, CDR2 and CDR3 domains having the sequences set forth in SEQ ID NOs: 40, 41, and 42, respectively. In another embodiment, the antibody comprises the VH region having the sequence set forth in SEQ ID NO: 43, and the VL region having the sequence set forth in SEQ ID NO: 44.

Another exemplary anti-C5 antibody is described in Fukuzawa T. et al. (Sci. Rep. 7:1080, 2017). In another embodiment, the antibody, or antigen binding fragment thereof, comprises a heavy chain comprising SEQ ID NO: 45 and a light chain comprising SEQ ID NO: 46.

Another exemplary anti-C5 antibody is described in US20170355757. In one embodiment, the antibody comprises a heavy chain variable region comprising SEQ ID NO:47 and a light chain variable region comprising SEQ ID NO:48. In another embodiment, the antibody comprises a heavy chain comprising SEQ ID NO:49 and a light chain comprising SEQ ID NO:50.

In another embodiment, the antibody competes for binding with, and/or binds to the same epitope on C5 as, the above-mentioned antibodies. In another embodiment, the antibody has at least about 90% variable region amino acid sequence identity with the above-mentioned antibodies (e.g., at least about 90%, 91%, 92%, 93%, 94%, 95%, 96%, 97%, 98% or 99% variable region identity).

In one embodiment, the anti-C5 antibody, or antigen binding fragment, is administered at a fixed dose. For example, in one embodiment, the anti-C5 antibody, or antigen binding fragment, is administered at a dose of 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, or 11000 mg, without regard to the patient's weight.

In another embodiment, the anti-C5 antibody, or antigen binding fragment thereof, (e.g., ravulizumab) is administered at a dose of 2400 mg, 2700 mg, 3000 mg, 3300 mg, or 3600 mg.

In another embodiment, 600 mg of the anti-C5 antibody, or antigen binding fragment thereof, (e.g., eculizumab) is administered to the patient. In another embodiment, 600 mg of the anti-C5 antibody, or antigen binding fragment thereof, (e.g., eculizumab) is administered to the patient once weekly. In another embodiment, 900 mg of the anti-C5 antibody, or antigen binding fragment thereof, (e.g., eculizumab) is administered to the patient. In another embodiment, 900 mg of the anti-C5 antibody, or antigen binding fragment thereof, (e.g., eculizumab) is administered to the patient once every two weeks. In another embodiment, 600 mg of the anti-C5 antibody, or antigen binding fragment thereof, (e.g., eculizumab) is administered to the patient once weekly for four consecutive weeks, followed by administration of 900 mg of the anti-C5 antibody, or antigen binding fragment thereof, (e.g., eculizumab) every two weeks thereafter. In another embodiment, 600 mg of the anti-C5 antibody, or antigen binding fragment thereof, is administered to the patient on Days 1, 8, 15, and 22, followed by 900 mg of the anti-C5 antibody, or antigen binding fragment thereof, (e.g., eculizumab) on Day 19 every two weeks thereafter.

In another embodiment, the dose of the anti-C5 antibody, or antigen binding fragment, is based on the weight of the patient. For example, in one embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, or 11000 mg of the anti-C5 antibody, or antigen binding fragment is administered to a patient weighing ≥40 to <60 kg. In another embodiment, 2400 mg or 3000 mg of the anti-C5 antibody, or antigen binding fragment thereof, (e.g., ravulizumab) is administered to a patient weighing ≥40 to <60 kg. In another embodiment, 2400 mg or 3000 mg of the anti-C5 antibody, or antigen binding fragment thereof, (e.g., ravulizumab) is administered to a patient weighing ≥40 to <60 kg every two weeks. In another embodiment, 2400 mg or 3000 mg of the anti-C5 antibody, or antigen binding fragment thereof, (e.g., ravulizumab) is administered to a patient weighing ≥40 to <60 kg every eight weeks.

In another embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, or 11000 mg of the anti-C5 antibody, or antigen binding fragment thereof, is administered to a patient weighing ≥60 to <100 kg. In another embodiment, 2700 mg or 3300 mg of the anti-C5 antibody, or antigen binding fragment thereof, (e.g., ravulizumab) is administered to a patient weighing ≥60 to <100 kg. In another embodiment, 2700 mg or 3300 mg of the anti-C5 antibody, or antigen binding fragment thereof, (e.g., ravulizumab) is administered to a patient weighing ≥60 to <100 kg every two weeks. In another embodiment, 2700 mg or 3300 mg of the anti-C5 antibody, or antigen binding fragment thereof, (e.g., ravulizumab) is administered to a patient weighing ≥60 to <100 kg every eight weeks.

In another embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, or 11000 mg of the anti-C5 antibody, or antigen binding fragment, is administered to a patient weighing ≥100 kg. In another embodiment, 3000 mg or 3600 mg of the anti-C5 antibody, or antigen binding fragment thereof, (e.g., ravulizumab) is administered to a patient weighing ≥100 kg. In another embodiment, 3000 mg or 3600 mg of the anti-C5 antibody, or antigen binding fragment thereof, (e.g., ravulizumab) is administered to a patient weighing ≥100 kg every two weeks. In another embodiment, 3000 mg or 3600 mg of the anti-C5 antibody, or antigen binding fragment thereof, (e.g., ravulizumab) is administered to a patient weighing ≥100 kg every eight weeks.

In another embodiment, a method of treating a human patient with vitiligo is provided, the method comprising administering to the patient an anti-C5 antibody, or antigen binding fragment thereof (e.g., eculizumab or ravulizumab):

(a) once on Day 1 at a dose of: 2400 mg to a patient weighing ≥40 to <60 kg, 2700 mg to a patient weighing ≥60 to <100 kg, or 3000 mg to a patient weighing ≥100 kg; and

(b) on Day 15 and every eight weeks thereafter at a dose of 3000 mg to a patient weighing ≥40 to <60 kg, 3300 mg to a patient weighing ≥60 to <100 kg, or 3600 mg to a patient weighing ≥100 kg.

In another embodiment, a method of treating a human patient with vitiligo is provided, wherein the anti-C5 antibody, or antigen binding fragment thereof, (e.g., eculizumab or ravulizumab) is administered to a patient weighing ≥40 to <60 kg:

(a) once on Day 1 at a dose of 2400 mg; and

(b) on Day 15 and every eight weeks thereafter at a dose of

3000 mg.

In another embodiment, a method of treating a human patient with vitiligo is provided, wherein the anti-C5 antibody, or antigen binding fragment thereof, (e.g., eculizumab or ravulizumab) is administered to a patient weighing ≥60 to <100 kg:

(a) once on Day 1 at a dose of 2700 mg; and

(b) on Day 15 of the administration cycle and every eight weeks thereafter at a dose of

3300 mg.

In another embodiment, a method of treating a human patient with vitiligo is provided, wherein the anti-C5 antibody, or antigen binding fragment thereof, (e.g., eculizumab or ravulizumab) is administered to a patient weighing ≥100 kg:

(a) once on Day 1 at a dose of 3000 mg; and

(b) on Day 15 and every eight weeks thereafter at a dose of

3600 mg.

In another embodiment, the anti-C5 antibody, or antigen binding fragment, is administered at a milligram per kilogram (mg/kg) dose. For example, in one embodiment, the anti-C5 antibody, or antigen binding fragment thereof, is administered at a dose of 0.1 mg/kg, 0.25 mg/kg, 0.5 mg/kg, 0.75 mg/kg, 1.0 mg/kg, 1.25 mg/kg, 1.50 mg/kg, 1.75 mg/kg, 2.0 mg/kg, 2.25 mg/kg, 2.50 mg/kg, 2.75 mg/kg, 3.0 mg/kg, 3.25 mg/kg, 3.50 mg/kg, 3.75 mg/kg, 4.0 mg/kg, 4.25 mg/kg, 4.50 mg/kg, 4.75 mg/kg, 5.0 mg/kg, 5.25 mg/kg, 5.50 mg/kg, 5.75 mg/kg, 6.0 mg/kg, 6.25 mg/kg, 6.50 mg/kg, 6.75 mg/kg, 7.0 mg/kg, 7.25 mg/kg, 7.50 mg/kg, 7.75 mg/kg, 8.0 mg/kg, 8.25 mg/kg, 8.50 mg/kg, 8.75 mg/kg, 9.0 mg/kg, 9.25 mg/kg, 9.50 mg/kg, 9.75 mg/kg, 10.0 mg/kg, 11.25 mg/kg, 11.50 mg/kg, 11.75 mg/kg, 12.0 mg/kg, 12.25 mg/kg, 12.50 mg/kg, 12.75 mg/kg, 13.0 mg/kg, 13.25 mg/kg, 13.50 mg/kg, 13.75 mg/kg, 14.0 mg/kg, 14.25 mg/kg, 14.50 mg/kg, 14.75 mg/kg, 15.0 mg/kg, 15.25 mg/kg, 15.50 mg/kg, 15.75 mg/kg, 16.0 mg/kg, 16.25 mg/kg, 16.50 mg/kg, 16.75 mg/kg, 17.0 mg/kg, 17.25 mg/kg, 17.50 mg/kg, 17.75 mg/kg, 18.0 mg/kg, 18.25 mg/kg, 18.50 mg/kg, 18.75 mg/kg, 19.0 mg/kg, 19.25 mg/kg, 19.50 mg/kg, 19.75 mg/kg, 20.0 mg/kg, 20.25 mg/kg, 20.50 mg/kg, 20.75 mg/kg, 21.0 mg/kg, 21.25 mg/kg, 21.50 mg/kg, 21.75 mg/kg, 22.0 mg/kg, 22.25 mg/kg, 22.50 mg/kg, 22.75 mg/kg, 23.0 mg/kg, 23.25 mg/kg, 23.50 mg/kg, 23.75 mg/kg, 24.0 mg/kg, 24.25 mg/kg, 24.50 mg/kg, 24.75 mg/kg, or 25.0 mg/kg.

In one embodiment, the anti-C5 antibody, or antigen binding fragment, is administered once per week, twice per week, three times per week, four times per week, five times per week, six times per week, or daily. In another embodiment, the anti-C5 antibody, or antigen binding fragment, is administered twice daily. In another embodiment, anti-C5 antibody, or antigen binding fragment, is administered once every two weeks, once every three weeks, once every four weeks, once every five weeks, once every six weeks, once every seven weeks, once every eight weeks, once every nine weeks, once every ten weeks, once every eleven weeks, or once every twelve weeks. In another embodiment, the anti-C5 antibody, or antigen binding fragment, is administered at a loading dose on Day 1, followed by a different maintenance dose on Day 15 and every eight weeks thereafter.

In another embodiment, the anti-C5 antibody, or antigen binding fragment thereof, is administered for one or more administration cycles. In one embodiment, the administration cycle is 26 weeks. In another embodiment, the treatment comprises at least 1, 2, 3, 4, 5, 6, 7, 8, 9, 10, or 11 cycles. In another embodiment, the treatment continues for the lifetime of the human patient.

In some embodiments, the patient has not previously been treated with a complement inhibitor (e.g., the patient is a complement inhibitor treatment-naïve patient).

The anti-C5 antibody, or antigen binding fragment, can be administered via any suitable means. In one embodiment, the anti-C5 antibody, or antigen binding fragment, is administered intravenously. In another embodiment, the anti-C5 antibody, or antigen binding fragment, is administered subcutaneously.

In another aspect, methods of treating a human patient with vitiligo are provided, the method comprising administering to the patient an effective amount of a first anti-C5 antibody, or antigen binding fragment thereof, followed by a second anti-C5 antibody or antigen-binding fragment. In one embodiment, the patient has previously been treated with one anti-C5 antibody, or antigen binding fragment thereof, and is switched to another anti-C5 antibody during the course of treatment. For example, in one embodiment, the patient is treated with eculizumab, followed by treatment with another anti-C5 antibody. In another embodiment, the patient is treated with ravulizumab, followed by treatment with another anti-C5 antibody.

In another embodiment, wherein a patient is treated with a first anti-C5 antibody and then switched to treatment with a second different anti-C5 antibody, especially where the second different anti-C5 antibody binds to a different epitope on C5 than the first anti-C5 antibody, the administration schedules takes into account the half-life of the first anti-C5 antibody. For example, to ensure that the first anti-C5 antibody is cleared (e.g., “washed out”) from the patient before the second (different) anti-C5 antibody is administered (e.g., to avoid issues associated with aggregation, immune complex formation, etc.), the half-life of the first anti-C5 antibody is taken into consideration. In one embodiment, the second (different) anti-C5 antibody is not administered until a duration of time corresponding to 2, 2.5, 3, 3.5, 4, 4.5, 5, 5.5, 6, 6.5, 7, or 7.5 times the half-life of the first anti-C5 antibody has passed after the final administration of the first anti-C5 antibody.

In another embodiment, the patient has previously been treated with eculizumab and then is switched to treatment with a second (different) anti-C5 antibody. In one embodiment where eculizumab is the first administered antibody, the second (different) anti-C5 antibody is not administered, for example, until at least 36, 45, 54, 63, 72, 81, 90, 99, 108, 117, or 126 days have passed after the final administration of eculizumab.

In another embodiment, the patient has previously been treated with ravulizumab and then is switched to treatment with a different anti-C5 antibody. In one embodiment where ravulizumab is the first administered antibody, the second (different) anti-C5 antibody is not administered, for example, until at least 100, 125, 150, 175, 200, 225, 250, 275, 300, 325, 375, or 400 days have passed after the final administration of ravulizumab.

Additionally, or alternatively, techniques are used to clear or enhance clearance of the first anti-C5 antibody before switching to treatment with a second (different) anti-C5 antibody. Exemplary techniques include, but are not limited to, plasmapheresis or blood transfusions. In another embodiment, an antibody against the first anti-C5 antibody is administered to clear or enhance clearance of the first anti-C5 antibody before a second (different) anti-C5 antibody is administered.

In some embodiments, the patients treated according to the methods described herein have been vaccinated against meningococcal infections within 3 years prior to, or at the time of, initiating treatment. In one embodiment, patients who received treatment less than 2 weeks after receiving a meningococcal vaccine are also treated with appropriate prophylactic antibiotics until 2 weeks after vaccination. In another embodiment, patients treated according to the methods described herein are vaccinated against meningococcal serotypes A, C, Y, W135, and/or B.

In another aspect, the treatment regimens described are sufficient to maintain particular serum trough concentrations of the anti-C5 antibody, or antigen binding fragment thereof. For example, in one embodiment, the treatment can maintain a serum trough concentration of the anti-C5 antibody, or antigen binding fragment thereof, of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395, or 400 μg/mL or greater. In one embodiment, the treatment maintains a serum trough concentration of the anti-C5 antibody, or antigen binding fragment thereof, of 100 μg/mL or greater. In another embodiment, the treatment maintains a serum trough concentration of the anti-C5 antibody, or antigen binding fragment thereof, of 150 μg/mL or greater. In another embodiment, the treatment maintains a serum trough concentration of the anti-C5 antibody, or antigen binding fragment thereof, of 200 μg/mL or greater. In another embodiment, the treatment maintains a serum trough concentration of the anti-C5 antibody, or antigen binding fragment thereof, of 250 μg/mL or greater. In another embodiment, the treatment maintains a serum trough concentration of the anti-C5 antibody, or antigen binding fragment thereof, of 300 μg/mL or greater. In another embodiment, the treatment maintains a serum trough concentration of the anti-C5 antibody, or antigen binding fragment thereof, of between 100 μg/ml and 200 μg/mL. In another embodiment, the treatment maintains a serum trough concentration of the anti-C5 antibody, or antigen binding fragment thereof, of about 175 μg/mL.

In another embodiment, to obtain an effective response, the anti-C5 antibody is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, or 260 μg of antibody per milliliter of the patient's blood. In another embodiment, the anti-C5 antibody is administered to the patient in an amount and with a frequency to maintain between 50 μg and 250 μg of antibody per milliliter of the patient's blood. In another embodiment, the anti-C5 antibody is administered to the patient in an amount and with a frequency to maintain between 100 μg and 200 μg of antibody per milliliter of the patient's blood. In another embodiment, the anti-C5 antibody is administered to the patient in an amount and with a frequency to maintain about 175 μg of antibody per milliliter of the patient's blood.

In another embodiment, to obtain an effective response, the anti-C5 antibody is administered to the patient in an amount and with a frequency to maintain a minimum free C5 concentration. For example, in one embodiment, the anti-C5 antibody can be administered to the patient in an amount and with a frequency to maintain a free C5 concentration of 0.2 μg/mL, 0.3 μg/mL, 0.4 μg/mL, 0.5 μg/mL or below. In another embodiment, the anti-C5 antibody is administered to the patient in an amount and with a frequency to maintain a free C5 concentration of 0.309 to 0.5 μg/mL or below. In another embodiment, the treatment described herein reduces free C5 concentration by greater than 99% throughout the treatment period. In another embodiment, the treatment reduces free C5 concentration greater than 99.5% throughout the treatment period.

In another aspect, the methods of treating vitiligo described herein can be used alone or in combination with one more additional therapies and/or therapeutic agents. For example, in one embodiment, the treatment further comprises administration of one or more of the following: topical or systemic corticosteroids, calcineurin inhibitors, and/or vitamin D analogues. In another embodiment, the treatment further comprises phototherapy.

The efficacy of the treatment methods provided herein can be assessed using any suitable means. In one embodiment, the treatment results in repigmentation. In another embodiment, repigmentation is assessed based on the percentage of repigmentation by one or more dermatologists by comparing pre-treatment and post-treatment photographs. In another embodiment, the treatment results in excellent repigmentation (ER) (>75% repigmentation), good repigmentation (GR) (50-75% repigmentation), or moderate repigmentation (MR) (25-50% repigmentation). In another embodiment, the treatment results in a Vitiligo Noticeability Scale (VNS) of 4 or 5.

Further provided are kits for treating vitiligo. In one embodiment, the kit comprises (a) a dose of an anti-C5 antibody, or antigen binding fragment thereof (e.g., any of those previously described herein); and (b) instructions for using the anti-C5 antibody, or antigen binding fragment thereof, in the methods described herein. In another embodiment, the kit is used to treat vitiligo and Paroxysmal Nocturnal Hemoglobinuria (PNH).

### BRIEF DESCRIPTION OF THE DRAWING

The FIGURE is a schematic that depicts the overall study design, treatments and study durations for ALXN1210-PNH-301, a Phase 3, open-label, randomized, active-controlled, multicenter study to evaluate the safety and efficacy of ravulizumab versus eculizumab administered by intravenous (IV) infusion to adult patients with PNH who are naïve to complement inhibitor treatment.

### DETAILED DESCRIPTION

### I. Anti-C5 Antibodies

The anti-C5 antibodies described herein bind to complement component C5 (e.g., human C5) and inhibit the cleavage of C5 into fragments C5a and C5b. As described above, such antibodies also have, for example, improved pharmacokinetic properties relative to other anti-C5 antibodies (e.g., eculizumab) used for therapeutic purposes.

The term “antibody” describes polypeptides comprising at least one antibody derived antigen binding site (e.g., V_{H}/V_{L} region or Fᵥ, or CDR). Antibodies include known forms of antibodies. An antibody can be, for example, a human antibody, a humanized antibody, a bispecific antibody or a chimeric antibody. An antibody also can be a Fab, Fab′2, ScFv, SMIP, Affibody®, nanobody or a domain antibody. An antibody also can be of any of the following isotypes: IgG1, IgG2, IgG3, IgG4, IgM, IgA1, IgA2, IgAsec, IgD, IgE or a hybrid of any of these isotypes. An antibody can be a naturally occurring antibody or an antibody that has been altered by a protein engineering technique (e.g., by mutation, deletion, substitution, conjugation to a non-antibody moiety). An antibody can include, for example, one or more variant amino acids (compared to a naturally occurring antibody), which changes a property (e.g., a functional property) of the antibody. Numerous such alterations are known in the art that affect, e.g., half-life, effector function, and/or immune responses to the antibody in a patient. The term “antibody” also includes artificial or engineered polypeptide constructs that comprise at least one antibody-derived antigen binding site.

Anti-C5 antibodies (or V_{H}/V_{L} domains derived therefrom) suitable for use herein can be generated using methods known in the art. Alternatively, art-recognized anti-C5 antibodies can be used. Antibodies that compete with any of these art-recognized antibodies for binding to C5 also can be used.

Eculizumab (also known as SOLIRIS®) is an anti-C5 antibody comprising heavy chain CDR1, CDR2 and CDR3 domains having the sequences set forth in SEQ ID NOs:1, 2 and 3, respectively, and light chain CDR1, CDR2 and CDR3 domains having the sequences set forth in SEQ ID NOs:4, 5 and 6, respectively. Eculizumab comprises a heavy chain variable region having the amino acid sequence set forth in SEQ ID NO:7 and a light chain variable region having the amino acid sequence set forth in SEQ ID NO:8. The variable regions of eculizumab are described in PCT/US1995/005688 and U.S. Pat. No. 6,355,245, the teachings of which are hereby incorporated by reference in their entirety. Eculizumab comprises a heavy chain comprising the amino acid sequence set forth in SEQ ID NO:10 and a light chain having the amino acid sequence set forth in SEQ ID NO:11. The full heavy and light chains of eculizumab are described in PCT/US2007/006606, the entire teachings of which are hereby incorporated by reference.

An exemplary anti-C5 antibody is ravulizumab comprising heavy and light chains having the sequences shown in SEQ ID NOs:14 and 11, respectively, or antigen binding fragments and variants thereof. Ravulizumab (also known as ULTOMIRIS®) is described in PCT/US2015/019225 and U.S. Pat. No. 9,079,949, the entire teachings of which are hereby incorporated by reference. Ravulizumab selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of ravulizumab. The antibody can comprise, for example, the CDR1, CDR2 and CDR3 domains of the V_{H} region of ravulizumab having the sequence set forth in SEQ ID NO:12, and the CDR1, CDR2 and CDR3 domains of the V_{L} region of ravulizumab having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1, CDR2 and CDR3 domains having the sequences set forth in SEQ ID NOs:4, 5 and 6, respectively. In another embodiment, the antibody comprises V_{H} and V_{L} regions having the amino acid sequences set forth in SEQ ID NO:12 and SEQ ID NO:8, respectively.

Another exemplary anti-C5 antibody comprises heavy and light chains having the sequences shown in SEQ ID NOs:20 and 11, respectively, or antigen binding fragments and variants thereof. In other embodiments, the antibody can comprise the heavy and light chain CDRs of SEQ ID Nos:20 and 11. Accordingly, in one embodiment, the antibody comprises the CDR1, CDR2 and CDR3 domains of the V_{H} having the sequence set forth in SEQ ID NO:12, and the CDR1, CDR2 and CDR3 domains of the V_{L} region having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1, CDR2 and CDR3 domains having the sequences set forth in SEQ ID NOs:4, 5 and 6, respectively.

The exact boundaries of CDRs have been defined differently according to different methods. In some embodiments, the positions of the CDRs or framework regions within a light or heavy chain variable domain can be as defined by Kabat et al. (“Sequences of Proteins of Immunological Interest.” NIH Publication No. 91-3242, U.S. Department of Health and Human Services, Bethesda, Md., 1991). In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia et al. (Nature, 342:877-83, 1989). Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs” (Thomas et al., Mol. Immunol., 33:1389-401, 1996).

In another embodiment, the antibody comprises V_{H} and V_{L} regions having the amino acid sequences set forth in SEQ ID NO:12 and SEQ ID NO:8, respectively. In another embodiment, the antibody comprises a heavy chain constant region as set forth in SEQ ID NO:13. In another embodiment, the antibody comprises a heavy chain polypeptide as set forth in SEQ ID NO:14 and a light chain polypeptide as set forth in SEQ ID NO:11. In another embodiment, the antibody comprises a variant human Fc region that binds to human neonatal Fc receptor (FcRn), wherein the variant human Fc CH3 region comprises Met429Leu and Asn435Ser substitutions at residues corresponding to methionine 428 and asparagine 434 of a native human IgG Fc region, each in EU numbering.

In another embodiment, the antibody comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a variant human Fc region that binds to human neonatal Fc receptor (FcRn), wherein the variant human Fc CH3 region comprises Met429Leu and Asn435Ser substitutions at residues corresponding to methionine 428 and asparagine 434 of a native human IgG Fc region, each in EU numbering.

In another embodiments, an anti-C5 antibody described herein comprises a heavy chain CDR1 comprising, or consisting of, the following amino acid sequence: GHIFSNYWIQ (SEQ ID NO:19). In another embodiment, an anti-C5 antibody described herein comprises a heavy chain CDR2 comprising, or consisting of, the following amino acid sequence: EILPGSGHTEYTENFKD (SEQ ID NO:18).

In another embodiment, the antibody binds to human C5 at pH 7.4 and 25 C with an affinity dissociation constant (K_{D}) that is in the range 0.1 nM≤K_{D}≤1 nM. In another embodiment, the antibody binds to human C5 at pH 6.0 and 25 C with a K_{D}≥10 nM. In yet another embodiment, the K_{D} of the antibody or antigen-binding fragment thereof for human C5 at pH 6.0 at 25 C)/(K_{D} of the antibody or antigen-binding fragment thereof for human C5 at pH 7.4 at 25 C of the antibody is greater than 25.

Another exemplary anti-C5 antibody is as described in U.S. Pat. Nos. 8,241,628 and 8,883,158. In one embodiment, the antibody, or antigen binding fragment thereof, comprises heavy chain CDR1, CDR2 and CDR3 domains having the sequences set forth in SEQ ID NOs: 21, 22 and 23, respectively, and light chain CDR1, CDR2 and CDR3 domains having the sequences set forth in SEQ ID NOs:24, 25 and 26, respectively. In another embodiment, the antibody, or antigen binding fragment thereof, comprises the V_{H} region having the sequence set forth in SEQ ID NO:27, and the VL region having the sequence set forth in SEQ ID NO:28.

Another exemplary anti-C5 antibody is also described in U.S. Pat. Nos. 8,241,628 and 8,883,158. In one embodiment, the antibody, or antigen binding fragment thereof, comprises heavy chain CDR1, CDR2 and CDR3 domains having the sequences set forth in SEQ ID NOs:29, 30 and 31, respectively, and light chain CDR1, CDR2 and CDR3 domains having the sequences set forth in SEQ ID NOs:32, 33 and 34, respectively. In another embodiment, the antibody comprises the V_{H} region having the sequence set forth in SEQ ID NO:35, and the V_{L} region having the sequence set forth in SEQ ID NO:36.

Another exemplary anti-C5 antibody is described in US2016/0176954A1. In one embodiment, the antibody, or antigen binding fragment thereof, comprises heavy chain CDR1, CDR2 and CDR3 domains having the sequences set forth in SEQ ID NOs:37, 38 and 39, respectively, and light chain CDR1, CDR2 and CDR3 domains having the sequences set forth in SEQ ID NOs:40, 41 and 42, respectively. In another embodiment, the antibody comprises the V_{H} region having the sequence set forth in SEQ ID NO:43, and the V_{L} region having the sequence set forth in SEQ ID NO:44.

Another exemplary anti-C5 antibody is described in Fukuzawa T. et al. (Sci. Rep., 7:1080, 2017). In one embodiment, the antibody or antigen binding fragment thereof comprises a heavy chain comprising SEQ ID NO:45 and a light chain comprising SEQ ID NO:46.

Another exemplary anti-C5 antibody is described in US2017/0355757. In one embodiment, the antibody comprises a heavy chain variable region comprising SEQ ID NO:47 and a light chain variable region comprising SEQ ID NO:48. In another embodiment, the antibody comprises a heavy chain comprising SEQ ID NO:49 and a light chain comprising SEQ ID NO:50.

The antibodies described herein can compete for binding with, and/or bind to the same epitope on C5 as any of the above-mentioned antibodies. The term “binds to the same epitope” with reference to two or more antibodies means that the antibodies bind to the same segment of amino acid residues, as determined by a given method. Techniques for determining whether antibodies bind to the “same epitope on C5” with the antibodies described herein include, for example, epitope mapping methods, such as, X-ray analysis of crystals of antigen:antibody complexes that provides atomic resolution of the epitope and hydrogen/deuterium exchange mass spectrometry (HDX-MS). Other methods monitor the binding of the antibody to peptide antigen fragments or variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same V_{H} and V_{L} or the same CDR1, CDR2 and CDR3 sequences are expected to bind to the same epitope.

Antibodies described herein can have, for example, at least about 90% variable region amino acid sequence identity with the above-mentioned antibodies (e.g., at least about 90%, 91%, 92%, 93%, 94%, 95%, 96%, 97%, 98% or 99% variable region identity).

An anti-C5 antibody described herein can, in some embodiments, comprise a variant human Fc region that binds to human neonatal Fc receptor (FcRn) with greater affinity than that of the native human Fc region from which the variant human Fc region was derived. The Fc constant region can comprise, for example, one or more (e.g., two, three, four, five, six, seven, eight or more) amino acid substitutions relative to the native human Fc region from which the variant human Fc region was derived. The substitutions can increase the binding affinity of an IgG antibody containing the variant Fc region to FcRn at pH 6.0, while maintaining the pH dependence of the interaction. Methods for testing whether one or more substitutions in the Fc region of an antibody increase the affinity of the Fc region for FcRn at pH 6.0 (while maintaining pH dependence of the interaction) are known in the art and exemplified in the working examples.

Substitutions that enhance the binding affinity of an antibody Fc region for FcRn are known in the art and include, e.g., (1) the M252Y/S254T/T256E triple substitution (Dall'Acqua, W. et al., J. Biol. Chem., 281:23514-24, 2006); (2) the M428L or T250Q/M428L substitutions (Hinton, P. et al., J. Biol. Chem., 279:6213-6, 2004; Hinton, P. et al., J. Immunol., 176:346-56); and (3) the N434A or T307/E380A/N434A substitutions (Petkova, S. et al., Int. Immunol., 18:1759-69, 2006). Additional substitution pairings, for example, P257I/Q3111, P257I/N434H and D376V/N434H are described in, e.g., Datta-Mannan, A. et al. (J. Biol. Chem., 282:1709-17, 2007). The disclosures of each of these references are incorporated herein by reference in its entireties.

In some embodiments, the constant region can comprise a substitution at EU amino acid residue 255 for valine, a substitution at EU amino acid residue 309 for asparagine, a substitution at EU amino acid residue 312 for isoleucine and/or a substitution at EU amino acid residue 386.

The antibod(ies) described herein can comprise a variant Fc region of no more than 30 (e.g., no more than 29, 28, 27, 26, 25, 24, 23, 22, 21, 20, 19, 18, 17, 16, 15, 14, 13, 12, 11, 10, 9, 8, 7, 6, 5, 4, 3 or 2) amino acid substitutions, insertions or deletions relative to the native constant region from which it was derived. In some embodiments, the variant Fc region comprises one or more amino acid substitutions selected from the group consisting of: M252Y, S254T, T256E, N434S, M428L, V259I, T250I and V308F. In some embodiments, the variant human Fc region comprises a methionine at position 428 and an asparagine at position 434, each in EU numbering. In some embodiments, the variant Fc region comprises a 428L/434S double substitution as described in, e.g., U.S. Pat. No. 8,088,376.

In some embodiments the precise location of substitutions can be shifted from the native human Fc region position as desired for antibody engineering. For example, the 428L/434S double substitution, when used in a IgG2/4 chimeric Fc, can correspond to 429L and 435S as in the M429L and N435S variants found in ravulizumab.

An antibody described herein can comprise, for example, a constant region comprising a substitution at one or more amino acid positions 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434 or 436 (EU numbering) relative to the native human constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan or tyrosine for threonine at position 250; phenylalanine, tryptophan or tyrosine for methionine at position 252; threonine for serine at position 254; glutamic acid for arginine at position 255; aspartic acid, glutamic acid or glutamine for threonine at position 256; alanine, glycine, isoleucine, leucine, methionine, asparagine, serine, threonine or valine for proline at position 257; histidine for glutamic acid at position 258; alanine for aspartic acid at position 265; phenylalanine for aspartic acid at position 270; alanine or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan or tyrosine for threonine at position 307; alanine, phenylalanine, isoleucine, leucine, methionine, proline, glutamine or threonine for valine at position 308; alanine, aspartic acid, glutamic acid, proline or arginine for leucine or valine at position 309; alanine, histidine or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan or tyrosine for methionine at position 428; lysine for histidine at position 433; alanine, phenylalanine, histidine, serine, tryptophan or tyrosine for asparagine at position 434; and histidine for tyrosine or phenylalanine at position 436, all in EU numbering.

Suitable anti-C5 antibodies for use in the methods described herein, in some embodiments, comprise a heavy chain polypeptide comprising the amino acid sequence depicted in SEQ ID NO:14 and/or a light chain polypeptide comprising the amino acid sequence depicted in SEQ ID NO:11. Alternatively, the anti-C5 antibodies for use in the methods described herein, in some embodiments, comprise a heavy chain polypeptide comprising the amino acid sequence depicted in SEQ ID NO:20 and/or a light chain polypeptide comprising the amino acid sequence depicted in SEQ ID NO:11.

In one embodiment, the antibody binds to C5 at pH 7.4 and 25 C (and, otherwise, under physiologic conditions) with a K_{D} that is at least 0.1 nM (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95 or 0.975 nM). In some embodiments, the K_{D} of the anti-C5 antibody, or antigen binding fragment thereof, is no greater than 1 nM (e.g., no greater than 0.9, 0.8, 0.7, 0.6, 0.5, 0.4, 0.3, or 0.2 nM).

In other embodiments, the K_{D} of the antibody for C5 at pH 6.0 at 25 C)/(K_{D} of the antibody for C5 at pH 7.4 at 25 C is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500 or 8000).

An anti-C5 antibody described herein can have a serum half-life in humans that is, for example, at least 20 days (e.g., at least 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55 days). In another embodiment, the anti-C5 antibody described herein has a serum half-life in humans that is at least 40 days. In another embodiment, the anti-C5 antibody described herein has a serum half-life in humans that is approximately 43 days. In another embodiment, the anti-C5 antibody described herein has a serum half-life in humans that is between 39-48 days. Methods for measuring the serum half-life of an antibody are known in the art. In some embodiments, an anti-C5 antibody, or antigen binding fragment thereof, described herein has a serum half-life that is at least 20% greater than the serum half-life of eculizumab (e.g., at least 30, 35, 40, 45, 50, 55, 60, 65, 70, 75, 80, 85, 90, 95, 100, 125, 150, 175, 200, 250, 300, 400 or 500% greater than the serum half-life of eculizumab), as measured, for example, in one of the mouse model systems described in the working examples (e.g., the C5-deficient/NOD/scid mouse or hFcRn transgenic mouse model system).

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody.” Competing antibodies bind to the same epitope, an overlapping epitope or to adjacent epitopes (e.g., as evidenced by steric hindrance).

Anti-C5 antibodies or antigen-binding fragments thereof described herein, used in the methods described herein can be generated using a variety of art-recognized techniques. Monoclonal antibodies can be obtained by various techniques familiar to those skilled in the art. Briefly, spleen cells from an animal immunized with a desired antigen are commonly immortalized by fusion with a myeloma cell (Köhler, G. & Milstein, C., Eur. J. Immunol., 6:511-9, 1976). Alternative methods of immortalization include transformation with Epstein Barr Virus, oncogenes or retroviruses, or other methods known in the art. Colonies arising from single immortalized cells are screened for production of antibodies of the desired specificity and affinity for the antigen, and yield of the monoclonal antibodies produced by such cells can be enhanced by various techniques, including injection into the peritoneal cavity of a vertebrate host. Alternatively, one can isolate DNA sequences that encode a monoclonal antibody or a binding fragment thereof by screening a DNA library from human B cells (Huse, W. et al., Science, 246:1275-81, 1989).

### II. Compositions

Compositions comprising an anti-C5 antibody or antigen binding fragment thereof, as described herein, can be formulated as a pharmaceutical solution. Pharmaceutical compositions generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. Compositions can include, for example, a pharmaceutically acceptable salt, e.g., an acid addition salt or a base addition salt, sugars, carbohydrates, polyols and/or tonicity modifiers.

Compositions described herein can be formulated according to standard methods. Pharmaceutical formulation is a well-established art (Gennaro, “Remington: The Science and Practice of Pharmacy,” 20^{th} Edition, Lippincott, Williams & Wilkins (ISBN: 0683306472), 2000; Ansel et al., “Pharmaceutical Dosage Forms and Drug Delivery Systems,” 7^{th} Edition, Lippincott Williams & Wilkins Publishers (ISBN: 0683305727), 1999; and Kibbe, “Handbook of Pharmaceutical Excipients American Pharmaceutical Association,” 3^{rd} Edition (ISBN: 091733096X), 2000). In some embodiments, a composition can be formulated, for example, as a buffered solution at a suitable concentration and suitable for storage at 2-8 C (e.g., 4 C). In some embodiments, a composition can be formulated for storage at a temperature below 0 C (e.g., −20 C or −80 C). In some embodiments, the composition can be formulated for storage for up to 2 years (e.g., 1 month, 2 months, 3 months, 4 months, 5 months, 6 months, 7 months, 8 months, 9 months, 10 months, 11 months, 1 year, 1½ years or 2 years) at 2-8 C (e.g., 4 C). Thus, in some embodiments, the compositions described herein are stable in storage for at least 1 year at 2-8 C (e.g., 4 C).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. Compositions containing a composition intended for systemic or local delivery, for example, can be in the form of injectable or infusible solutions. Accordingly, the compositions can be formulated for administration by a parenteral mode (e.g., intravenous, subcutaneous, intraperitoneal or intramuscular injection). “Parenteral administration,” “administered parenterally,” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

In one embodiment, the composition comprises eculizumab for injection. In another embodiment, the composition comprises ravulizumab for injection. In one embodiment, the injection is a sterile, clear to translucent, slightly whitish color, preservative-free solution for intravenous use. In another embodiment, each single-dose vial contains 300 mg ravulizumab for injection at a concentration of 10 mg/mL with a pH of 7.0. In another embodiment, ravulizumab for injection requires dilution to a final concentration of 5 mg/mL. In another embodiment, each mL further comprises polysorbate 80 (0.2 mg; vegetable origin), sodium chloride (8.77 mg), sodium phosphate dibasic (1.78 mg), sodium phosphate monobasic (0.46 mg) and water.

### III. Methods of Treatment

Provided herein are methods for treating vitiligo (e.g., refractory vitiligo) in a human patient, comprising administering to the patient an anti-C5 antibody, or antigen binding fragment thereof. As used herein, the term “subject” or “patient” is a human patient (e.g., a patient having vitiligo).

Vitiligo is an acquired skin disorder affecting 0.5-2% of the population worldwide, characterized by solitary or multiple well-defined depigmented macules (Ezzedine, K. et al., JAMA, 316:1708-9, 2016; Fenniche, S. et al., Dermatol. Ther. (Heidelb.), 8:127-35, 2018). Vitiligo causes the loss of skin color in blotches. The extent and rate of color loss from vitiligo is unpredictable and can affect the skin on any part of the body, hair and/or the inside of the mouth. Normally, the color of hair and skin is determined by melanin. Vitiligo occurs when the cells that produce melanin die or stop functioning.

There are several types of vitiligo. Generalized vitiligo covers many parts of the body and the discolored patches often progress similarly on corresponding body parts (symmetrically). Segmental vitiligo is vitiligo where the discolored patches are on one side or part of the body. Segmental vitiligo tends to occur at a younger age, progress for a year or two, then stop. Localized (focal) vitiligo occurs on only a few areas of the body.

The main symptom of vitiligo is patchy loss of skin color. Discoloration usually first shows on sun-exposed areas, such as the hands, feet, arms, face and lips. Vitiligo signs can also include: patchy loss of skin color, premature whitening or graying of the hair on the scalp, eyelashes, eyebrows or beard, the loss of color in the tissues that line the inside of the mouth and nose (mucous membranes), and/or loss of or change in color of the inner layer of the eyeball (retina).

Treating vitiligo can restore color to the affected skin, but it does not prevent continued loss of skin color or a recurrence. Moreover, treating vitiligo is a challenge for dermatologists since there is no definitive curative treatment. Existing treatment options for vitiligo, include topical or systemic corticosteroids, calcineurin inhibitors, vitamin D analogues, systemic therapies, and surgery (Halder, R. et al., Dermatol. Clin., 18:79-89, 2000). Phototherapy, however, is the cornerstone of treating vitiligo. It modulates the immune response and induces activation of hair follicle and epidermal melanocyte precursors by ultraviolet (UV) light. Conventional phototherapy is available as broadband UVB (290-320 nm) or more commonly narrowband UVB (311-313 nm). Targeted phototherapy is particularly indicated in patients with localized vitiligo. The monochromatic excimer light (308 nm) delivers higher fluence to the depigmented lesions while sparing the normal skin (Leone, G. et al., J. Eur. Acad. Dermatol. Venereol., 17:531-7, 2003; Park, K. et al., Br. J. Dermatol., 167:468-78, 2012). Hair follicle is the main reservoir that supplies melanocytes in repigmenting human vitiligo. UV exposure stimulates proliferation and migration of melanocyte precursors along the infundibulum outer root sheath to the interfollicular epidermis (Goldstein, N. et al., J. Invest. Dermatol., 135:2068-76, 2015). In addition, Khellin has been used to treat vitiligo in association with UV (Anstey, A., J. Dermatol. Treat., 9:63-4, 1998).

In some embodiments, the patient has vitiligo and Paroxysmal Nocturnal Hemoglobinuria (PNH). PNH is an acquired hemolytic disorder that occurs most frequently in adults (Brodsky, R., Blood, 126:2459-65, 2015). The disease begins with the clonal expansion of a hematopoietic stem cell that has acquired a somatic mutation in the PIGA gene (Brodsky, R., Blood, 124:2804-11, 2014). Consequently, PNH blood cells lack the glycophosphatidylinositol (GPI) anchor protein and are deficient in the membrane-bound complement inhibitory proteins CD55 and CD59. In the absence of CD55, there is increased deposition of complement protein C3 cleavage products on blood cell membrane surfaces, in turn leading to cleavage of C5 into C5a and C5b. The pathology and clinical presentations in patients with PNH are driven by uncontrolled terminal complement activation.

C5a is a potent anaphylatoxin, chemotactic factor, and cell-activating molecule that mediates multiple pro-inflammatory and pro-thrombotic activities (Matis, L. et al., Nat. Med., 1:839-42, 1995; Prodinger et al., Complement. In: Paul W E, editor. Fundamental immunology (4th ed). Philadelphia: Lippincott-Raven Publishers; 1999. p. 967-95). C5b recruits the terminal complement components C6, C7, C8 and C9 to form the pro-inflammatory, pro-thrombotic cytolytic pore molecule C5b-9, a process that under normal circumstances would be blocked on the red blood cell (RBC) membrane by CD59. In patients with PNH, however, these final steps proceed unchecked, culminating in hemolysis and the release of free hemoglobin, as well as platelet activation (Hill, A. et al., Blood, 121:4985-96, 2013). The signs and symptoms of PNH can be attributed to chronic, uncontrolled complement C5 cleavage, and release of C5a and C5b-9 leading to RBC hemolysis, which together result in release of intracellular free hemoglobin and lactate dehydrogenase (LDH) into circulation as a direct consequence of hemolysis, irreversible binding to and inactivation of nitric oxide (NO) by hemoglobin, and inhibition of NO synthesis, vasoconstriction and tissue-bed ischemia due to absence of vasodilatory NO, as well as possible microthrombi manifesting as abdominal pain, dysphagia, and erectile dysfunction, platelet activation, and/or pro-inflammatory and prothrombotic state. A substantial proportion of patients with PNH experience renal dysfunction and pulmonary hypertension (Hillmen, P. et al., Am. J. Hematol., 85:553-9, 2010 (erratum in Am. J. Hematol., 85:911, 2010)); Hill, A. et al., Br. J Haematol., 158:409-14, 2012). Patients also experience venous or arterial thrombosis in diverse sites, including the abdomen or central nervous system.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. In the context of PNH, for example, effective treatment may refer to alleviation of one more symptoms selected from the group consisting of fatigue, abdominal pain, dyspnea, dysphagia, chest pain, and/or erectile dysfunction).

In the context of vitiligo, for example, effective treatment results in repigmentation. In one embodiment, repigmentation is assessed based on the percentage of repigmentation by one or more dermatologists by comparing pre-treatment and post-treatment photographs. In another embodiment, the treatment results in excellent repigmentation (ER) (>75% repigmentation). In another embodiment, the treatment results in good repigmentation (GR) (50-75% repigmentation). In another embodiment, the treatment results in moderate repigmentation (MR) (25-50% repigmentation).

In another embodiment, effective treatment results in a Vitiligo Noticeability Scale (VNS) of 4 or 5. The VNS is a patient-rated outcome measure of vitiligo treatment response (Batchelor, J. et al., Br. J. Dermatol., 174: 386-94, 2016). The VNS asks patients: “compared with before treatment, how noticeable is the vitiligo now?” A score of 1 indicates the vitiligo is more noticeable. A score of 2 indicates the vitiligo is as noticeable. A score of 3 indicates the vitiligo is slightly less noticeable. A score of 4 indicates the vitiligo is a lot less noticeable. A score of 5 indicates the vitiligo is no longer noticeable. A score of 4 or 5 means that the treatment was effective.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In one example, an “effective amount” is the amount of anti-C5 antibody, or antigen binding fragment thereof, clinically proven to alleviate at least one symptom of vitiligo (e.g., results in partial or complete repigmentation). An effective amount can be administered in one or more administrations.

In one embodiment, the anti-C5 antibody, or antigen binding fragment, is administered at a fixed dose. For example, in one embodiment, the anti-C5 antibody, or antigen binding fragment, is administered at a dose of 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 40 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg or 11000 mg, without regard to the patient's weight.

In another embodiment, the anti-C5 antibody, or antigen binding fragment thereof, (e.g., eculizumab or ravulizumab) is administered at a dose of 2400 mg, 2700 mg, 3000 mg, 3300 mg or 3600 mg.

In another embodiment, 600 mg of the anti-C5 antibody, or antigen binding fragment thereof, (e.g., eculizumab or ravulizumab) is administered to the patient. In another embodiment, 600 mg of the anti-C5 antibody, or antigen binding fragment thereof, (e.g., eculizumab or ravulizumab) is administered to the patient once weekly. In another embodiment, 900 mg of the anti-C5 antibody, or antigen binding fragment thereof, (e.g., eculizumab or ravulizumab) is administered to the patient. In another embodiment, 900 mg of the anti-C5 antibody, or antigen binding fragment thereof, (e.g., eculizumab or ravulizumab) is administered to the patient once every two weeks. In another embodiment, 600 mg of the anti-C5 antibody, or antigen binding fragment thereof, (e.g., eculizumab or ravulizumab) is administered to the patient once weekly for four consecutive weeks, followed by administration of 900 mg of the anti-C5 antibody, or antigen binding fragment thereof, (e.g., eculizumab or ravulizumab) every two weeks thereafter. In another embodiment, 600 mg of the anti-C5 antibody, or antigen binding fragment thereof, is administered to the patient on Days 1, 8, 15, and 22, followed by 900 mg of the anti-C5 antibody, or antigen binding fragment thereof, (e.g., eculizumab or ravulizumab) on Day 19 every two weeks thereafter.

In another embodiment, the dose of the anti-C5 antibody, or antigen binding fragment, is based on the weight of the patient. For example, in one embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg or 11000 mg of the anti-C5 antibody, or antigen binding fragment is administered to a patient weighing ≥40 to <60 kg. In another embodiment, 2400 mg or 3000 mg of the anti-C5 antibody, or antigen binding fragment thereof, (e.g., eculizumab or ravulizumab) is administered to a patient weighing ≥40 to <60 kg. In another embodiment, 2400 mg or 3000 mg of the anti-C5 antibody, or antigen binding fragment thereof, (e.g., eculizumab or ravulizumab) is administered to a patient weighing ≥40 to <60 kg every two weeks. In another embodiment, 2400 mg or 3000 mg of the anti-C5 antibody, or antigen binding fragment thereof, (e.g., eculizumab or ravulizumab) is administered to a patient weighing ≥40 to <60 kg every eight weeks.

In another embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, or 11000 mg of the anti-C5 antibody, or antigen binding fragment thereof, is administered to a patient weighing ≥60 to <100 kg. In another embodiment, 2700 mg or 3300 mg of the anti-C5 antibody, or antigen binding fragment thereof, (e.g., eculizumab or ravulizumab) is administered to a patient weighing ≥60 to <100 kg. In another embodiment, 2700 mg or 3300 mg of the anti-C5 antibody, or antigen binding fragment thereof, (e.g., eculizumab or ravulizumab) is administered to a patient weighing ≥60 to <100 kg every two weeks. In another embodiment, 2700 mg or 3300 mg of the anti-C5 antibody, or antigen binding fragment thereof, (e.g., eculizumab or ravulizumab) is administered to a patient weighing ≥60 to <100 kg every eight weeks.

In another embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg or 11000 mg of the anti-C5 antibody, or antigen binding fragment, is administered to a patient weighing ≥100 kg. In another embodiment, 3000 mg or 3600 mg of the anti-C5 antibody, or antigen binding fragment thereof, (e.g., eculizumab or ravulizumab) is administered to a patient weighing ≥100 kg. In another embodiment, 3000 mg or 3600 mg of the anti-C5 antibody, or antigen binding fragment thereof, (e.g., eculizumab or ravulizumab) is administered to a patient weighing ≥100 kg every two weeks. In another embodiment, 3000 mg or 3600 mg of the anti-C5 antibody, or antigen binding fragment thereof, (e.g., eculizumab or ravulizumab) is administered to a patient weighing ≥100 kg every eight weeks.

In another embodiment, a method of treating a human patient with vitiligo is provided, the method comprising administering to the patient an anti-C5 antibody, or antigen binding fragment thereof (e.g., eculizumab or ravulizumab): (a) once on Day 1 at a dose of: 2400 mg to a patient weighing ≥40 to <60 kg, 2700 mg to a patient weighing ≥60 to <100 kg, or 3000 mg to a patient weighing ≥100 kg; and (b) on Day 15 and every eight weeks thereafter at a dose of 3000 mg to a patient weighing ≥40 to <60 kg, 3300 mg to a patient weighing ≥60 to <100 kg, or 3600 mg to a patient weighing ≥100 kg.

In another embodiment, a method of treating a human patient with vitiligo is provided, wherein the anti-C5 antibody, or antigen binding fragment thereof, (e.g., eculizumab or ravulizumab) is administered to a patient weighing ≥40 to <60 kg: (a) once on Day 1 at a dose of 2400 mg; and (b) on Day 15 and every eight weeks thereafter at a dose of 3000 mg.

In another embodiment, a method of treating a human patient with vitiligo is provided, wherein the anti-C5 antibody, or antigen binding fragment thereof, (e.g., eculizumab or ravulizumab) is administered to a patient weighing ≥60 to <100 kg: (a) once on Day 1 at a dose of 2700 mg; and (b) on Day 15 of the administration cycle and every eight weeks thereafter at a dose of 3300 mg.

In another embodiment, a method of treating a human patient with vitiligo is provided, wherein the anti-C5 antibody, or antigen binding fragment thereof, (e.g., eculizumab or ravulizumab) is administered to a patient weighing ≥100 kg: (a) once on Day 1 at a dose of 3000 mg; and (b) on Day 15 and every eight weeks thereafter at a dose of 3600 mg.

In another embodiment, the anti-C5 antibody, or antigen binding fragment, is administered at a milligram per kilogram (mg/kg) dose. For example, in one embodiment, the anti-C5 antibody, or antigen binding fragment thereof, is administered at a dose of 0.1 mg/kg, 0.25 mg/kg, 0.5 mg/kg, 0.75 mg/kg, 1.0 mg/kg, 1.25 mg/kg, 1.50 mg/kg, 1.75 mg/kg, 2.0 mg/kg, 2.25 mg/kg, 2.50 mg/kg, 2.75 mg/kg, 3.0 mg/kg, 3.25 mg/kg, 3.50 mg/kg, 3.75 mg/kg, 4.0 mg/kg, 4.25 mg/kg, 4.50 mg/kg, 4.75 mg/kg, 5.0 mg/kg, 5.25 mg/kg, 5.50 mg/kg, 5.75 mg/kg, 6.0 mg/kg, 6.25 mg/kg, 6.50 mg/kg, 6.75 mg/kg, 7.0 mg/kg, 7.25 mg/kg, 7.50 mg/kg, 7.75 mg/kg, 8.0 mg/kg, 8.25 mg/kg, 8.50 mg/kg, 8.75 mg/kg, 9.0 mg/kg, 9.25 mg/kg, 9.50 mg/kg, 9.75 mg/kg, 10.0 mg/kg, 11.25 mg/kg, 11.50 mg/kg, 11.75 mg/kg, 12.0 mg/kg, 12.25 mg/kg, 12.50 mg/kg, 12.75 mg/kg, 13.0 mg/kg, 13.25 mg/kg, 13.50 mg/kg, 13.75 mg/kg, 14.0 mg/kg, 14.25 mg/kg, 14.50 mg/kg, 14.75 mg/kg, 15.0 mg/kg, 15.25 mg/kg, 15.50 mg/kg, 15.75 mg/kg, 16.0 mg/kg, 16.25 mg/kg, 16.50 mg/kg, 16.75 mg/kg, 17.0 mg/kg, 17.25 mg/kg, 17.50 mg/kg, 17.75 mg/kg, 18.0 mg/kg, 18.25 mg/kg, 18.50 mg/kg, 18.75 mg/kg, 19.0 mg/kg, 19.25 mg/kg, 19.50 mg/kg, 19.75 mg/kg, 20.0 mg/kg, 20.25 mg/kg, 20.50 mg/kg, 20.75 mg/kg, 21.0 mg/kg, 21.25 mg/kg, 21.50 mg/kg, 21.75 mg/kg, 22.0 mg/kg, 22.25 mg/kg, 22.50 mg/kg, 22.75 mg/kg, 23.0 mg/kg, 23.25 mg/kg, 23.50 mg/kg, 23.75 mg/kg, 24.0 mg/kg, 24.25 mg/kg, 24.50 mg/kg, 24.75 mg/kg or 25.0 mg/kg.

In one embodiment, the anti-C5 antibody, or antigen binding fragment is administered once per week, twice per week, three times per week, four times per week, five times per week, six times per week or daily. In another embodiment, the anti-C5 antibody, or antigen binding fragment, is administered twice daily. In another embodiment, anti-C5 antibody, or antigen binding fragment, is administered once every two weeks, once every three weeks, once every four weeks, once every five weeks, once every six weeks, once every seven weeks, once every eight weeks, once every nine weeks, once every ten weeks, once every eleven weeks or once every twelve weeks. In another embodiment, the anti-C5 antibody, or antigen binding fragment, is administered at a loading dose on Day 1, followed by a different maintenance dose on Day 15 and every eight weeks thereafter.

In another embodiment, the anti-C5 antibody, or antigen binding fragment thereof, is administered for one or more administration cycles. In one embodiment, the administration cycle is 26 weeks. In another embodiment, the treatment comprises at least 1, 2, 3, 4, 5, 6, 7, 8, 9, 10 or 11 cycles. In another embodiment, the treatment continues for the lifetime of the human patient.

In some embodiments, the patient has not previously been treated with a complement inhibitor (e.g., the patient is a complement inhibitor treatment-naïve patient).

The anti-C5 antibody, or antigen binding fragment, can be administered via any suitable means. In one embodiment, the anti-C5 antibody, or antigen binding fragment, is administered intravenously. In another embodiment, the anti-C5 antibody, or antigen binding fragment, is administered subcutaneously.

In another aspect, methods of method of treating a human patient with vitiligo are provided, the method comprising administering to the patient an effective amount of a first anti-C5 antibody, or antigen-binding fragment thereof, followed by a second anti-C5 antibody or antigen-binding fragment. In one embodiment, the patient has previously been treated with one anti-C5 antibody, or antigen binding fragment thereof, and is switched to another anti-C5 antibody during the course of treatment. In one embodiment, for example, the patient is treated with eculizumab or ravulizumab, followed by treatment with another anti-C5 antibody (e.g., any of those described previously). In another embodiment, the patient is treated with ravulizumab, followed by treatment with another anti-C5 antibody (e.g., any of those described previously).

In another embodiment, when a patient is treated with a first anti-C5 antibody and then switched to treatment with a second different anti-C5 antibody, where the second different anti-C5 antibody binds to a different epitope on C5 than the first anti-C5 antibody, the administration schedules takes into account the half-life of the first anti-C5 antibody. Consideration is taken, for example, to ensure that the first anti-C5 antibody is cleared (e.g., “washed out”) from the patient before the second (different) anti-C5 antibody is administered (e.g., to avoid issues associated with aggregation, immune complex formation, etc.). In one embodiment, the second (different) anti-C5 antibody is not administered until a duration of time corresponding to 2, 2.5, 3, 3.5, 4, 4.5, 5, 5.5, 6, 6.5, 7 or 7.5 times the half-life of the first anti-C5 antibody has passed after the final administration of the first anti-C5 antibody.

In another embodiment, the patient has previously been treated with eculizumab and then is switched to treatment with a second (different) anti-C5 antibody (e.g., any of those described previously). In one embodiment where eculizumab is the first administered antibody, the second (different) anti-C5 antibody is not administered, for example, until at least 36, 45, 54, 63, 72, 81, 90, 99, 108, 117 or 126 days have passed after the final administration of eculizumab.

In another embodiment, the patient has previously been treated with ravulizumab and then is switched to treatment with a different anti-C5 antibody (e.g., any of those described previously). In one embodiment where ravulizumab is the first administered antibody, the second (different) anti-C5 antibody is not administered, for example, until at least 100, 125, 150, 175, 200, 225, 250, 275, 300, 325, 375 or 400 days have passed after the final administration of ravulizumab.

Additionally, or alternatively, techniques are used to clear or enhance clearance of the first anti-C5 antibody before switching to treatment with a second (different) anti-C5 antibody. Exemplary techniques include, but are not limited to, plasmapheresis or blood transfusions. In another embodiment, an antibody against the first anti-C5 antibody is administered to clear or enhance clearance of the first anti-C5 antibody before a second (different) anti-C5 antibody is administered.

In some embodiments, the patients treated according to the methods described herein have been vaccinated against meningococcal infections within three years prior to, or at the time of, initiating treatment. In one embodiment, patients who received treatment less than two weeks after receiving a meningococcal vaccine are also treated with appropriate prophylactic antibiotics until two weeks after vaccination. In another embodiment, patients treated according to the methods described herein are vaccinated against meningococcal serotypes A, C, Y, W135 and/or B.

In another aspect, the treatment regimens described are sufficient to maintain particular serum trough concentrations of the anti-C5 antibody, or antigen binding fragment thereof. The treatment can maintain, for example, a serum trough concentration of the anti-C5 antibody, or antigen binding fragment thereof, of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395 or 400 μg/mL or greater. In one embodiment, the treatment maintains a serum trough concentration of the anti-C5 antibody, or antigen binding fragment thereof, of 100 μg/mL or greater. In another embodiment, the treatment maintains a serum trough concentration of the anti-C5 antibody, or antigen binding fragment thereof, of 150 μg/mL or greater. In another embodiment, the treatment maintains a serum trough concentration of the anti-C5 antibody, or antigen binding fragment thereof, of 200 μg/mL or greater. In another embodiment, the treatment maintains a serum trough concentration of the anti-C5 antibody, or antigen binding fragment thereof, of 250 μg/mL or greater. In another embodiment, the treatment maintains a serum trough concentration of the anti-C5 antibody, or antigen binding fragment thereof, of 300 μg/mL or greater. In another embodiment, the treatment maintains a serum trough concentration of the anti-C5 antibody, or antigen binding fragment thereof, of between 100 μg/mL and 200 μg/mL. In another embodiment, the treatment maintains a serum trough concentration of the anti-C5 antibody, or antigen binding fragment thereof, of about 175 μg/mL.

In another embodiment, to obtain an effective response, the anti-C5 antibody is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg or 260 μg of antibody per milliliter of the patient's blood. In another embodiment, the anti-C5 antibody is administered to the patient in an amount and with a frequency to maintain between 50 μg and 250 μg of antibody per milliliter of the patient's blood. In another embodiment, the anti-C5 antibody is administered to the patient in an amount and with a frequency to maintain between 100 μg and 200 μg of antibody per milliliter of the patient's blood. In another embodiment, the anti-C5 antibody is administered to the patient in an amount and with a frequency to maintain about 175 μg of antibody per milliliter of the patient's blood.

In another embodiment, to obtain an effective response, the anti-C5 antibody is administered to the patient in an amount and with a frequency to maintain a minimum free C5 concentration. The anti-C5 antibody can be administered, for example, to the patient in an amount and with a frequency to maintain a free C5 concentration of 0.2 μg/mL, 0.3 μg/mL, 0.4 μg/mL, 0.5 μg/mL or below. In another embodiment, the anti-C5 antibody is administered to the patient in an amount and with a frequency to maintain a free C5 concentration of 0.309 to 0.5 μg/mL or below. In another embodiment, the treatment described herein reduces free C5 concentration by greater than 99% throughout the treatment period. In another embodiment, the treatment reduces free C5 concentration greater than 99.5% throughout the treatment period.

In another aspect, the methods of treating vitiligo described herein can be used alone or in combination with one more additional therapies and/or therapeutic agents. For example, in one embodiment, the treatment further comprises administration of one or more of the following: topical or systemic corticosteroids, calcineurin inhibitors and/or vitamin D analogues. In another embodiment, the treatment further comprises phototherapy.

### IV. Kits and Unit Dosage Forms

Also provided herein are kits that include a pharmaceutical composition containing an anti-C5 antibody, or antigen binding fragment thereof (e.g., any of those described herein previously) and a pharmaceutically acceptable carrier, in a therapeutically effective amount adapted for use in the methods described herein. The kits optionally also can include instructions, e.g., comprising administration schedules, to allow a practitioner (e.g., a physician, nurse or patient) to administer the composition contained therein to administer the composition to a patient having vitiligo. The kit also can include a syringe.

Optionally, the kits include multiple packages of the single-dose pharmaceutical compositions each containing an effective amount of the anti-C5 antibody, or antigen binding fragment thereof, for a single administration in accordance with the methods provided above. Instruments or devices necessary for administering the pharmaceutical composition(s) also may be included in the kits. For instance, a kit may provide one or more pre-filled syringes containing an amount of the anti-C5 antibody, or antigen binding fragment thereof.

The following example is merely illustrative and should not be construed as limiting the scope of this disclosure in any way as many variations and equivalents will become apparent to those skilled in the art upon reading the present disclosure. The contents of all references, patents and published patent applications cited throughout this application are expressly incorporated herein by reference.

### EXAMPLE

### Example 1: Complement Inhibitor-Naïve Adult Patient with PNH and Refractory Vitiligo Treated with Eculizumab, Followed by Ravulizumab

A Phase 3, randomized, open-label, active-controlled study of ravulizumab versus eculizumab was conducted in complement inhibitor-naïve adult patients with paroxysmal nocturnal hemoglobinuria (PNH) as described in PCT/US2018/057760, the contents of which are expressly incorporated herein by reference. The overall study design is depicted in the FIGURE. In the phase 3 study, patients randomized to ravulizumab received a loading dose (2400 mg for patients ≥40 kg to <60 kg, 2700 mg for patients ≥60 kg to <100 kg, 3000 mg for patients ≥100 kg) on day 1, followed by maintenance doses of ravulizumab (3000 mg for patients ≥40 kg to <60 kg, 3300 mg for patients ≥60 to <100 kg, 3600 mg for patients ≥100 kg) on day 15 and q8w thereafter.

One patient included in this study was a 46-year-old male with a 25-year history of generalized vitiligo who was diagnosed with paroxysmal nocturnal hemoglobinuria (PNH). Months after diagnosis, he developed esophageal spasm and progressive hemolytic anemia, and commenced anti-complement therapy as a participant in the phase 3 clinical trial. He was treated with eculizumab and received a 600 mg loading dose weekly for four weeks, followed by eleven maintenance doses of 900 mg at two-week intervals. Afterwards, he received a 2700 mg loading dose of ravulizumab, followed by ongoing maintenance doses of 3300 mg at eight-week intervals.

Within four weeks of starting treatment with eculizumab, his symptoms resolved and his hemolytic indices normalized. Six months after starting therapy, the patient also unexpectedly noticed macules of repigmentation on his face and hands. He was on no active treatment for his vitiligo, which had been refractory to topical therapy, and he never had such an improvement in his vitiligo before starting eculizumab.

SEQUENCE SUMMARY

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCARY
FFGSSPNWYFDVWGQGTLVTVSS
SEQ ID NO: 8
DIQMTQSPSSLSASVGDRVTITCGASENIYGALNWYQQKPGKAPKWYGA
TNLADGVPSRFSGSGSGTDFTLTISSLQPEDFATYYCQNVLNTPLTFGQGTK
VEIK
SEQ ID NO: 9
ASTKGPSVFPLAPCSRSTSESTAALGCLVKDYFPEPVTVSWNSGALTSGVH
TFPAVLQSSGLYSLSSVVTVPSSNFGTQTYTCNVDHKPSNTKVDKTVERKC
CVECPPCPAPPVAGPSVFLFPPKPKDTLMISRTPEVTCVVVDVSQEDPEVQF
NWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCS
VMHEALHNHYTQKSLSLSLGK
SEQ ID NO: 10
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCAR
YFFGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGTQTYT
CNVDHKPSNTKVDKTVERKCCVECPPCPAPPVAGPSVFLFPPKPKDTLMISR
TPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTV
DKSRWQEGNVFSCSVMHEALHNHYTQKSLSLSLGK
SEQ ID NO: 11
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPV
TKSFNRGEC
SEQ ID NO: 12
QVQLVQSGAEVKKPGASVKVSCKASGHIFSNYWIQWVRQAPGQGLEW
MGEILPGSGHTEYTENFKDRVTMTRDTSTSTVYMELSSLRSEDTAVYYC
ARYFFGSSPNWYFDVWGQGTLVTVSS
SEQ ID NO: 13
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDPE
VQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVLHEALHSHYTQKSLSLSLGK
SEQ ID NO: 14
QVQLVQSGAEVKKPGASVKVSCKASGHIFSNYWIQWVRQAPGQGLEWM
GEILPGSGHTEYTENFKDRVTMTRDTSTSTVYMELSSLRSEDTAVYYCAR
YFFGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGTQTYT
CNVDHKPSNTKVDKTVERKCCVECPPCPAPPVAGPSVFLFPPKPKDTLMISR
TPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTV
DKSRWQEGNVFSCSVLHEALHSHYTQKSLSLSLGK
SEQ ID NO: 15
ASTKGPSVFPLAPCSRSTSESTAALGCLVKDYFPEPVTVSWNSGALTSGVH
TFPAVLQSSGLYSLSSVVTVTSSNFGTQTYTCNVDHKPSNTKVDKTVERKC
CVECPPCPAPPVAGPSVFLFPPKPKDTLYITREPEVTCVVVDVSHEDPEVQF
NWYVDGMEVHNAKTKPREEQFNSTFRVVSVLTVVHQDWLNGKEYKCKV
SNKGLPAPIEKTISKTKGQPREPQVYTLPPSREEMTKNQVSLTCLVKGFYP
SDIAVEWESNGQPENNYKTTPPMLDSDGSFFLYSKLTVDKSRWQQGNVF
SCSVMHEALHNHYTQKSLSLSPGK
SEQ ID NO: 16
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCAR
YFFGSSPNWYFDVWGQGTLVTVSSASTKGPSVFPLAPCSRSTSESTAALG
CLVKDYFPEPVTVSWNSGALTSGVHTFPAVLQSSGLYSLSSVVTVTSSNF
GTQTYTCNVDHKPSNTKVDKTVERKCCVECPPCPAPPVAGPSVFLFPPKP
KDTLYITREPEVTCVVVDVSHEDPEVQFNWYVDGMEVHNAKTKPREEQ
FNSTFRVVSVLTVVHQDWLNGKEYKCKVSNKGLPAPIEKTISKTKGQPRE
PQVYTLPPSREEMTKNQVSLTCLVKGFYPSDIAVEWESNGQPENNYKTTP
PMLDSDGSFFLYSKLTVDKSRWQQGNVFSCSVMHEALHNHYTQKSLSLS
PGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAEVKKPGASVKVSCKASGHIFSNYWIQWVRQAPGQGLEW
MGEILPGSGHTEYTENFKDRVTMTRDTSTSTVYMELSSLRSEDTAVYYC
ARYFFGSSPNWYFDVWGQGTLVTVSSASTKGPSVFPLAPCSRSTSESTAALG
CLVKDYFPEPVTVSWNSGALTSGVHTFPAVLQSSGLYSLSSVVTVPSSNFGTQT
YTCNVDHKPSNTKVDKTVERKCCVECPPCPAPPVAGPSVFLFPPKPKDTLMIS
RTPEVTCVVVDVSQEDPEVQFNWYVDGVEVHNAKTKPREEQFNSTYRVVSVL
TVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTV
DKSRWQEGNVFSCSVMHEALHNHYTQKSLSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGAEVKKPGSSVKVSCKASGGTFSSYAISVWRQAPGQGLEWMGGIGPF
FGTANYAQKFQGRVTITADESTSTAYMELSSLRSEDTAVYYCARDTPYFD
YWGQGTLVTVSS
SEQ ID NO: 28
DIELTQPPSVSVAPGQTARISCSGDSIPNYYVYWYQQKPGQAPVLVIYDDSNRPSG
IPERFSGSNSGNTATLTISGTQAEDEADYYCQSFDSSLNAEVFGGGTKLTVL
SEQ ID NO: 29
NYIS
SEQ ID NO: 30
IIDPDDSYTEYSPSFQG
SEQ ID NO: 31
YEYGGFDI
SEQ ID NO: 32
SGDNIGNSYVH
SEQ ID NO: 33
KDNDRPS
SEQ ID NO: 34
GTYDIESYV
SEQ ID NO: 35
EVQLVQSGAEVKKPGESLKISCKGSGYSFTNYISWVRQMPGKGLEWMGIIDPDDS
YTEYSPSFQGQVTISADKSISTAYLQWSSLKASDTAMYYCARYEYGGFDI
WGQGTLVTVSS
SEQ ID NO: 36
SYELTQPPSVSVAPGQTARISCSGDNIGNSYVHWYQQKPGQAPVLVIYKDNDRPS
GIPERFSGSNSGNTATLTISGTQAEDEADYYCGTYDIESYVFGGGTKLTVL
SEQ ID NO: 37
SSYYVA
SEQ ID NO: 38
AIYTGSGATYKASWAKG
SEQ ID NO: 39
DGGYDYPTHAMHY
SEQ ID NO: 40
QASQNIGSSLA
SEQ ID NO: 41
GASKTHS
SEQ ID NO: 42
QSTKVGSSYGNH
SEQ ID NO: 43
QVQLVESGGGLVQPGGSLRLSCAASGFTSHSSYYVAWVRQAPGKGLEWVGAIYT
GSGATYKASWAKGRFTISKDTSKNQVVLTMTNMDPVDTATYYCASDGGYDYPT
HAMHYWGQGTLVTVSS
SEQ ID NO: 44
DVVMTQSPSSLSASVGDRVTITCQASQNIGSSLAWYQQKPGQAPRLLIYGASKTH
SGVPSRFSGSGSGTDFTLTISSLQPEDVATYYCQSTKVGSSYGNHFGGGTKVEIK
SEQ ID NO: 45
QVQLVESGGGLVQPGRSLRLSCAASGFTVHSSYYMAWVRQAPGKGLEWVGAIF
TGSGAEYKAEWAKGRVTISKDTSKNQVVLTMTNMDPVDTATYYCASDAGYDYP
THAMHYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTK
VDKKVEPKSCDKTHTCPPCPAPELRRGPKVFLFPPKPKDTLMISRTPEVTCVVVDV
SHEDPEVKFNWYVDGVEVHNAKTKPREEQYNSTYRVVSVLTVLHQDWLNGKEY
KCKVSNKGLPSSIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVLHE
ALHAHYTRKELSLSP
SEQ ID NO: 46
DIQMTQSPSSLSASVGDRVTITCRASQGISSSLAWYQQKPGKAPKLLIYGASETES
GVPSRFSGSGSGTDFTLTISSLQPEDFATYYCQNTKVGSSYGNTFGGGTKVEIKRT
VAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
SEQ ID NO: 47
QVQLQESGPGLVKPSETLSLTCTVSGDSVSSSYWTWIRQPPGKGLEWIGYIYYSGS
SNYNPSLKSRATISVDTSKNQFSLKLSSVTADTAVYYCAREGNVDTTMIFDYWG
QGTLVTVSS
SEQ ID NO: 48
AIQMTQSPSSLSASVGDRVTITCRASQGIRNDLGWYQQKPGKAPKLLIYAASSLQS
GVPSRFAGRGSGTDFTLTISSLQPEDFATYYCLQDFNYPWTFGQGTKVEIK
SEQ ID NO: 49
QVQLQESGPGLVKPSETLSLTCTVSGDSVSSSYWTWIRQPPGKGLEWIGYIYYSGS
SNYNPSLKSRATISVDTSKNQFSLKLSSVTAADTAVYYCAREGNVDTTMIFDYWG
QGTLVTVSSASTKGPSVFPLAPCSRSTSESTAALGCLVKDYFPEPVTVSWNSGALT
SGVHTFPAVLQSSGLYSLSSVVTVPSSSLGTKTYTCNVDHKPSNTKVDKRVESKY
GPPCPPCPAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHYTQKSL
SLSLGK
SEQ ID NO: 50
AIQMTQSPSSLSASVGDRVTITCRASQGIRNDLGWYQQKPGKAPKLLIYAASSLQS
GVPSRFAGRGSGTDFTLTISSLQPEDFATYYCLQDFNYPWTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC

What is claimed is:

1. A method of treating a human patient with vitiligo, the method comprising administering to the patient an effective amount of an anti-C5 antibody or antigen binding fragment thereof.

2. The method of claim 1, wherein the anti-C5 antibody or antigen binding fragment thereof comprises CDR1, CDR2, and CDR3 heavy chain sequences as set forth in SEQ ID NOs:1, 2 and 3, respectively, and CDR1, CDR2 and CDR3 light chain sequences as set forth in SEQ ID NOs:4, 5 and 6, respectively.

3. The method of claim 1 or 2, wherein the anti-C5 antibody or antigen binding fragment thereof comprises a heavy chain variable region as set forth in SEQ ID NO:7 and a light chain variable region as set forth in SEQ ID NO:8.

4. The method of any one of the preceding claims, wherein the anti-C5 antibody or antigen binding fragment thereof, comprises a heavy chain as set forth in SEQ ID NO:10 and a light chain as set forth in SEQ ID NO:11.

5. The method of claim 1, wherein the anti-C5 antibody or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively.

6. The method of claim 1 or 5, wherein the antibody comprises a variant human Fc region that binds to human neonatal Fc receptor (FcRn), wherein the variant human Fc CH3 region comprises Met-429-Leu and Asn-435-Ser substitutions at residues corresponding to methionine 428 and asparagine 434 of a native human IgG Fc region, each in EU numbering.

7. The method of any one of claims 1 and 5-6, wherein the anti-C5 antibody or antigen binding fragment thereof, comprises a heavy chain variable region as set forth in SEQ ID NO:12 and a light chain variable region as set forth in SEQ ID NO:8.

8. The method of any one of claims 1 and 5-7, wherein the anti-C5 antibody or antigen-binding fragment thereof, further comprises a heavy chain constant region depicted in SEQ ID NO:13.

9. The method of any one of claims 1 and 5-8, wherein the anti-C5 antibody or antigen binding fragment thereof comprises a heavy chain as set forth in SEQ ID NO:14 and a light chain as set forth in SEQ ID NO:11.

10. The method of any one of claims 1 and 5-9, wherein the anti-C5 antibody or antigen-binding fragment thereof binds to human C5 at pH 7.4 and 25 C with an affinity dissociation constant (K_{D}) that is in the range 0.1 nM≤K_{D}≤1 nM.

11. The method of any one of claims 1 and 5-10, wherein the anti-C5 antibody or antigen-binding fragment thereof binds to human C5 at pH 6.0 and 25 C with a K_{D}≥10 nM.

12. The method of claim 1, wherein, the anti-C5 antibody, or antigen-binding fragment thereof is selected from the group consisting of:
(i) an antibody, or antigen binding fragment thereof, comprising heavy chain CDR1, CDR2 and CDR3 domains comprising SEQ ID NOs:21, 22 and 23, respectively, and light chain CDR1, CDR2 and CDR3 domains comprising SEQ ID NOs:24, 25 and 26, respectively;
(ii) an antibody, or antigen binding fragment thereof, comprising a heavy chain variable region comprising SEQ ID NO:27 and a light chain variable region comprising SEQ ID NO:28;
(iii) an antibody, or antigen binding fragment thereof, comprising heavy chain CDR1, CDR2 and CDR3 domains comprising SEQ ID NOs:29, 30 and 31, respectively, and light chain CDR1, CDR2 and CDR3 domains comprising SEQ ID NOs:32, 33 and 34, respectively;
(iv) an antibody, or antigen binding fragment thereof, comprising a heavy chain variable region comprising SEQ ID NO:35 and a light chain variable region comprising SEQ ID NO:36;
(v) an antibody, or antigen binding fragment thereof, comprising heavy chain CDR1, CDR2 and CDR3 domains comprising SEQ ID NOs:37, 38 and 39, respectively, and light chain CDR1, CDR2 and CDR3 domains comprising SEQ ID NOs:40, 41 and 42, respectively;
(vi) an antibody, or antigen binding fragment thereof, comprising a heavy chain variable region comprising SEQ ID NO:43 and a light chain variable region comprising SEQ ID NO:44;
(vii) an antibody, or antigen binding fragment thereof, comprising a heavy chain comprising SEQ ID NO:45 and a light chain comprising SEQ ID NO:46;
(viii) an antibody, or antigen binding fragment thereof, comprising a heavy chain variable region comprising SEQ ID NO:47 and a light chain variable region comprising SEQ ID NO:48; and
(ix) an antibody, or antigen binding fragment thereof, comprising a heavy chain comprising SEQ ID NO:49 and a light chain comprising SEQ ID NO:50.

13. The method of any one of the preceding claims, wherein the patient also has Paroxysmal Nocturnal Hemoglobinuria (PNH).

14. The method of any one of claims 1-4, wherein 600 mg of the anti-C5 antibody or antigen binding fragment thereof is administered to the patient once weekly.

15. The method of any one of claims 1-4, wherein 900 mg of the anti-C5 antibody or antigen binding fragment thereof is administered to the patient once every two weeks.

16. The method any one of claims 1-4, wherein 600 mg of the anti-C5 antibody or antigen binding fragment thereof is administered to the patient once weekly for four consecutive weeks, followed by administration of 900 mg of the anti-C5 antibody or antigen binding fragment thereof every two weeks thereafter.

17. The method any one of claims 1-4, wherein 600 mg of the anti-C5 antibody or antigen binding fragment thereof is administered to the patient on Days 1, 8, 15 and 22, followed by 900 mg of the anti-C5 antibody or antigen binding fragment thereof on Day 19 every two weeks thereafter.

18. The method any one of claims 1 and 5-11, wherein the anti-C5 antibody, or antigen binding fragment thereof, is administered at a dose of 2400 mg, 2700 mg, 3000 mg, 3300 mg or 3600 mg.

19. The method any one of claims 1 and 5-11, wherein the anti-C5 antibody or antigen binding fragment thereof is administered at a dose of 2400 mg to a patient weighing ≥40 to <60 kg, 2700 mg to a patient weighing ≥60 to <100 kg, or 3000 mg to a patient weighing ≥100 kg.

20. The method of any one of claims 1, 5-11, and 18-19, wherein the anti-C5 antibody or antigen binding fragment thereof is administered to the patient every two weeks.

21. The method any one of claims 1 and 5-11, wherein the anti-C5 antibody or antigen binding fragment thereof is administered at a dose of 3000 mg to a patient weighing ≥40 to <60 kg, 3300 mg to a patient weighing ≥60 to <100 kg, or 3600 mg to a patient weighing ≥100 kg.

22. The method of any one of claims 1, 5-11, 18, and 21, wherein the anti-C5 antibody or antigen binding fragment thereof is administered to the patient every eight weeks.

23. The method any one of claims 1 and 5-11, wherein the anti-C5 antibody or antigen binding fragment thereof is administered:
(a) once on Day 1 at a dose of: 2400 mg to a patient weighing ≥40 to <60 kg, 2700 mg to a patient weighing ≥60 to <100 kg, or 3000 mg to a patient weighing ≥100 kg; and
(b) on Day 15 and every eight weeks thereafter at a dose of 3000 mg to a patient weighing ≥40 to <60 kg, 3300 mg to a patient weighing ≥60 to <100 kg, or 3600 mg to a patient weighing ≥100 kg.

24. The method of any one of claims 1 and 5-11, wherein the anti-C5 antibody or antigen binding fragment thereof is administered to a patient weighing ≥40 to <60 kg:
(a) once on Day 1 at a dose of 2400 mg; and
(b) on Day 15 and every eight weeks thereafter at a dose of 3000 mg.

25. The method of any one of claims 1 and 5-11, wherein the anti-C5 antibody or antigen binding fragment thereof is administered to a patient weighing ≥60 to <100 kg:
(a) once on Day 1 at a dose of 2700 mg; and
(b) on Day 15 of the administration cycle and every eight weeks thereafter at a dose of 3300 mg.

26. The method of any one of claims 1 and 5-11, wherein the anti-C5 antibody or antigen binding fragment thereof is administered to a patient weighing ≥100 kg:
(a) once on Day 1 at a dose of 3000 mg; and
(b) on Day 15 and every eight weeks thereafter at a dose of 3600 mg.

27. The method of any one of the preceding claims, wherein the anti-C5 antibody or antigen binding fragment thereof is formulated for intravenous administration.

28. A method of treating a human patient with vitiligo, the method comprising administering to the patient an effective amount of a first anti-C5 antibody or antigen-binding fragment thereof, followed by a second anti-C5 antibody or antigen-binding fragment thereof.

29. The method of claim 28, wherein the first anti-C5 antibody or antigen-binding fragment thereof is eculizumab and the second anti-C5 antibody or antigen-binding fragment is ravulizumab.

30. The method of claim 28 or 29, wherein the patient also has Paroxysmal Nocturnal Hemoglobinuria (PNH).

31. The method of any one of the preceding claims, wherein the treatment further comprises administration of one or more of the following: topical or systemic corticosteroids, calcineurin inhibitors and/or vitamin D analogues.

32. The method of any one of the preceding claims, wherein the treatment further comprises phototherapy.

33. The method of any one of the preceding claims, wherein the treatment results in repigmentation.

34. The method of claim 33, wherein the repigmentation is assessed based on the percentage of repigmentation by one or more dermatologists by comparing pre-treatment and post-treatment photographs.

35. The method of claim 33 or 34, wherein the treatment results in excellent repigmentation (ER) (>75% repigmentation), good repigmentation (GR) (50-75% repigmentation), or moderate repigmentation (MR) (25-50% repigmentation).

36. The method of any one of the preceding claims, wherein the treatment results in a Vitiligo Noticeability Scale (VNS) of 4 or 5.

37. A kit for treating vitiligo in a human patient, the kit comprising:
(a) a dose of an anti-C5 antibody or antigen binding fragment thereof; and
(b) instructions for using the anti-C5 antibody, or antigen binding fragment thereof, in the method of any one of claims 1-36.

38. The kit of claim 37, wherein the patient also has Paroxysmal Nocturnal Hemoglobinuria (PNH).
